(19) United States
(12) Patent Application Publication
(10) Pub. No.: US 2006/0025707 A1
(43) Pub. Date: Feb. 2, 2006

(54) Method and apparatus for evaluating motor nerve impairment in a patient suffering from lower lumber discopathy

(76) Inventor: Alex Finsterbush, Jerusalem (IL)

Correspondence Address: Martin Moynihan;c/o ANTHONY CASTORINA, 2001 JEFFERSON DAVIS HIGHWAY, SUITE 207, ARLINGTON, VA, 22202, US

(21) Appl. No.: 10/902,950

(22) Filed: Aug. 2, 2004

(51) Int. Cl. A61B 5/103

(52) U.S. Cl. 600/592

(57) ABSTRACT

A method and apparatus for evaluating motor nerve impairment in a patient suffering from lumber discopathy, by: immobilizing both lower legs and feet of the patient but permitting movement of the patient's two big toes; measuring the maximum forces capable of being applied simultaneously by the patient's two big toes in extension; and utilizing the measurements for evaluating the nerve impairment. The maximum forces are measured by force sensors which require very little displacement of the two big toes, preferably less than 1.0 mm, for the effective range of the measurement. The maximum forces capable of being applied by the patient's two big toes are measured in each of a plurality of separate actions over a predetermined time interval so as also to provide an indication of the degree of fatigue.

### FIELD AND BACKGROUND OF THE INVENTION

The present invention relates to a method and apparatus for evaluating motor nerve impairment in a patient, particularly in a patient suffering from lower lumber discopathy.

A major reason for low back pain is lumbar discopathy. Complaints for this condition include radicular pain to one or both lower limbs, lack of localized sensitivity in the foot, and deficient motor responses of muscles in the foot resulting in weakness or inability to perform certain movements.

Motor nerves of feet muscles originate in the lumbar roots. The slightest pressure on these roots can impairment, or decrease the response of, a number of motor units of the nerve. Therefore, motor weakness is one of the most sensitive criteria to detect pressure on the nerve root.

The most common disc lesion in the lumbar region takes place in the lower level of the L4-5 and L5-S1 discs. Such a lesion causes weakness of the EHL (Extensor Hallucis Longus) muscle.

Physical examination tests performed today for detecting lumbar discopathy include: measurement of asymmetry in the diameter of the hips or thighs; comparison of sensitivity to touch and prick of the two sides; comparison of knee and ankle reflexes in both limbs; comparison of the ability to perform certain movements under resistance in both limbs; and comparison of dorsiflexion of the big toes in both feet against resistance. Other tests include: electromyographic tests; somato-sensory evoked response; computerized tomography scans; and MRI.

Accuracy in neurologic evaluation of patients depends upon objective and quantitative tests, preferably performed in the physician's office. Various neurophysiological tests performed today in sophisticated research laboratories are not generally repeatable on follow-up examinations at the physician's office.

At the present time, evaluation of a patient with low-back problem is generally based mainly upon the subjective impression of the physician. However, such impressions frequently do not correlate with the actual condition of the patient as indicated by clinical findings, for the following reasons:

1. Mobility of the lumbar spine is limited by pain and not necessarily by organic pathology; mobility improves with analgesic drugs or physical treatment.

2. Interpretation of sensory deficit depends on the patient's cooperation and understanding, and is highly misleading and subjective.

3. Quantitative evaluation of motor deficit is extremely difficult because:

a. a decrease of motor response smaller than 30 to 40% from normal motor power can not be effectively detected by the physician during a clinical examination: as a result, an unknown number of patients with lumbar discopathy with a mild motor deficit are undiagnosed;

b. the examination is totally subjective and therefore various examiners may report motor deficit differently;

c. limitations, such as pain, in performing tests can be misinterpreted as motor weakness.

d. the patient's motivation can affect the test's results;

e. no objective measurement and reporting of test results are available for use in objective follow-up of the patient's condition;

f. no objective reference is available as to the severity of the illness.

An article in Spine, Volume 8, Nov. 6, 1983, pages 206-210, titled “Quantitative Power Measurement of Extensor Hallucis Longus” by A. Finsterbush, U. Frankel, and R. Arnon, describes an apparatus, previously developed by a team including the inventor in the present application, for providing a simple objective test in the evaluation of low-back pain with a neurological involvement. This apparatus is more particularly illustrated in FIG. 1, described below. However, the use of that apparatus in the evaluation of such back pains encountered several serious problems, as will also be described more particularly below with respect to FIG. 1.

### OBJECTS AND BRIEF SUMMARY OF THE PRESENT INVENTION

Broad objects of the present invention are to provide a method and apparatus based on the method and apparatus described in the above-cited A. Finsterbush et al publication but having a number of important advantages thereover.

According to one aspect of the present invention, there is provided a method of evaluating motor nerve impairment in a patient suffering from lower lumber discopathy, comprising: immobilizing both lower legs and feet of the patient but permitting movement of the patient's two big toes; measuring the maximum forces capable of being applied simultaneously by the patient's two big toes in extension; and utilizing said measurements for evaluating the nerve impairment.

According to further features in the preferred embodiment of the invention described below, the maximum forces are measured by force sensors which require very little displacement of the two big toes for the effective operating range of the measurement. Preferably, the force sensors are each displaceable for less than 1.0 mm for the effective range of the measurement. In the described preferred embodiment, the force sensors are compressible resistor elements which change their electrical resistances in accordance with the compression force applied.

According to another feature in the preferred embodiment of the invention described below, the maximum forces capable of being applied by the patient's two big toes are measured in each of a plurality of separate actions over a predetermined time interval of at least one minute.

According to still further features in the described preferred embodiment, the force sensors are force transducers which output electrical signals corresponding to the forces applied by the respective toes. The method also includes the additional operations of storing, processing and displaying the electrical signals outputted from the force sensors.

According to another aspect of the present invention, there is provided apparatus for evaluating motor nerve impairment in a patient suffering from lower lumber discopathy, comprising: a frame configured for receiving and immobilizing both lower legs and feet of the patient but permitting movement of the patient's two big toes; and two force sensors carried by the frame at locations such that each force sensor is aligned with and engaged by one of the big toes of the patient when the lower legs and feet of the patient are immobilized on the frame, for measuring the maximum forces capable of being simultaneously applied by the two big toes in extension.

According to a further aspect of the present invention, there is provided apparatus for evaluating nerve impairment in a patient suffering from lumber discopathy, comprising: a frame configured for receiving and immobilizing at least one lower leg and foot of the patient but permitting movement of the patient's two big toes; and a force sensor carried by the frame in alignment with, and engaged by, the big toe of the immobilized leg and foot of the patient for measuring the maximum force capable of being applied by the big toe in extension. The force sensor includes a base adjustably mounted on the frame to overlie the immobilized foot of the patient when the patient is in a reclining position. The apparatus further comprises an end wall rigidly secured to one end of the base to depend therefrom and to be located just forwardly of the big toe of the patient's immobilized foot; and a sensor pad carried on the inner face of the end wall in alignment with the big toe of the immobilized foot. The base is adjustably mounted to the frame by a lateral extension of the base formed with a slot receiving an adjusting screw enabling adjustment of the sensor pad with respect to the big toe of the patient's immobilized foot.

As will be described more particularly below, the method and apparatus of the present invention are capable of providing a number of important advantages in evaluating nerve impairment in a patient suffering from lumber discopathy as compared to the method and apparatus described in the above-cited A. Finsterbush et al publication.

Further features of the invention will be apparent from the description below.

### BRIEF DESCRIPTION OF THE DRAWINGS

The invention is herein described, by way of example only, with reference to the accompanying drawings, wherein:

FIG. 1 illustrates a prior art apparatus constructed in accordance with the description in the above-cited Finsterbush et al publication of 1983;

FIG. 2 illustrates one form of apparatus constructed in accordance with the present invention;

FIG. 3 is a block diagram of a data processor system included in the apparatus of FIG. 2;

FIG. 4 illustrates outputs of the force sensors in the apparatus of FIG. 2 when used with respect to a patient having a healthy right leg and a slightly problematic left leg;

FIG. 5 illustrates the outputs of the force sensors in one case (represented by curves A and B) when the two legs were tested concurrently, and in another case (curves A and C) when one leg (represented by curve C) was tested about ten minutes after the first leg (curve A);

FIG. 6 illustrates the outputs of the force sensors when both legs, tested concurrently, were found problematic, the condition of one leg (right) being more severe than in the other;

FIG. 7 illustrates the outputs of the force sensors when both legs were found to suffer from a fatigue condition;

FIGS. 8, 9 and 10 are side, end and plan views, respectively, illustrating another force sensor construction usable in the above-described apparatus; and

FIG. 11 illustrates the manner of using the force sensor of FIGS. 6-10 in the apparatus of FIG. 2.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

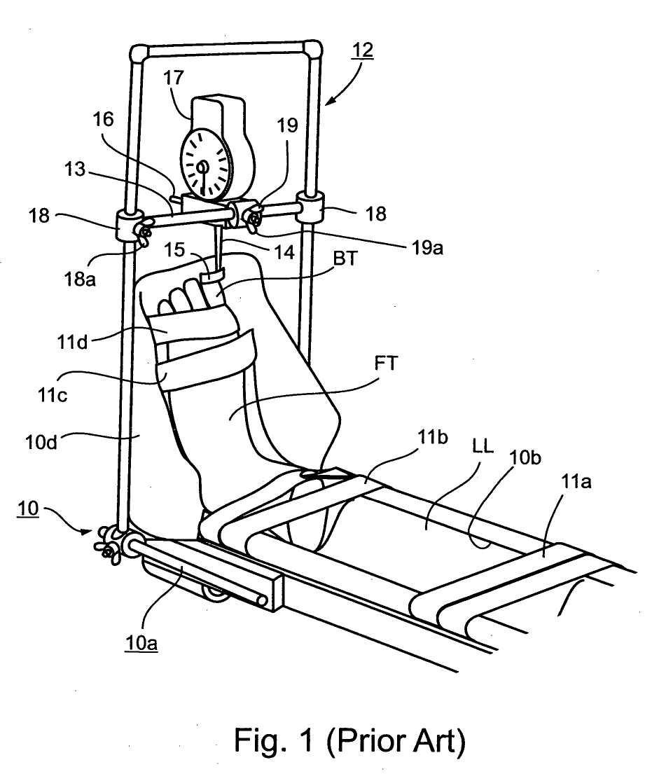

### The Prior Art Apparatus of FIG. 1

The prior art apparatus illustrated in FIG. 1, as more particularly described in the above-cited A. Finsterbush et al publication of 1983, includes a frame, generally designated 10, configured for receiving and immobilizing only one lower leg LL and the respective foot FT of the patient, while permitting movement of the big toe BT of the patient in the immobilized foot. Thus, the frame includes a horizontal section 10a formed with a channel 10b for receiving the lower leg LL of the patient, and an end wall 10c extending substantially perpendicularly to the channel for contacting the sole of the patient's foot FT when the patient's lower leg LL is received within channel 10b.

The apparatus illustrated in FIG. 1 further includes flexible straps 11a and 11b for binding the lower leg LL to the frame section 10a, flexible strap 11c for binding the foot FT to the end wall 10c, and flexible strap 11d for binding the toes, except for the big toe BT, firmly against the end wall 10c.

It will thus be seen that the big toe BT is not immobilized, but rather is permitted movement in extension, i.e., in the direction substantially perpendicular to, and away from, the sole of the respective foot.

The prior art apparatus illustrated in FIG. 1 further includes a vertically-extending rack 12 mounted to overlie end wall 10c, for mounting a force detector to detect the movements of the big toe BT. Thus, rack 12 mounts an adjustable horizontal bar 13 which in turn pivotally mounts a depending arm 14 in alignment with the big toe BT. Arm 14 carries at its outer end a curved contact element 15 engageable with the nail of the big toe BT. Arm 14 is coupled to a spring-type force transducer 16 which includes a gauge 17 to provide a visual indication of the force applied by the big toe BT, via contact element 15 and arm 14, to the force transducer 16.

Horizontal bar 13 is vertically adjustable by a pair of sleeves 18 carried at its opposite ends and slidable along rack 12. One sleeve 18 carries a wing nut 18a for fixing the horizontal bar 13 at the desired vertical position with respect to the big toe BT to be engaged by contact element 15 of the depending arm 14. Depending arm 14 is, in turn, horizontally adjustable by a sleeve 19 slidably movable along horizontal bar 13 and fixable thereto by another wing nut 19a.

The apparatus illustrated in FIG. 1 was used for evaluating motor nerve impairment by making separate measurements of the maximum force capable of being exerted by the big toe of one foot, and then by the big toe of the other foot. Such measurements of the actual force capabilities of each foot provided an indication of the Extensor Hallucis Longus (EHL) motor power for the respective foot. Such measurements were carried out in each of a plurality of separate actions over a predetermined time interval, e.g., two-three minutes, which thereby also provided an indication of the degree of fatigue suffered by the EHL muscle in the respective foot. Such information is extremely helpful in evaluating nerve impairment in a patient suffering from lumbar discopathy.

However, when the apparatus illustrated in FIG. 1, was used for evaluating nerve impairment by measuring (EHL) motor power, several serious problems were encountered: Since separate measurements were needed for the big toes on the two feet, not only was the examination time prolonged, but the patient's experience in the second test introduced an additional variable to the examination, in that fatigue in the first foot examination influenced the results in the second foot examination for the respective test. Moreover, since the examination required movement of the big toes in elderly patients with arthritic changes in their Hallux, the test produced pain and reduced cooperation, particularly when repeated motions were required. In addition, since the measurements were made mechanically and the results outputted only visually, the data was not recorded nor stored.

### A Preferred Embodiment of the Present Invention

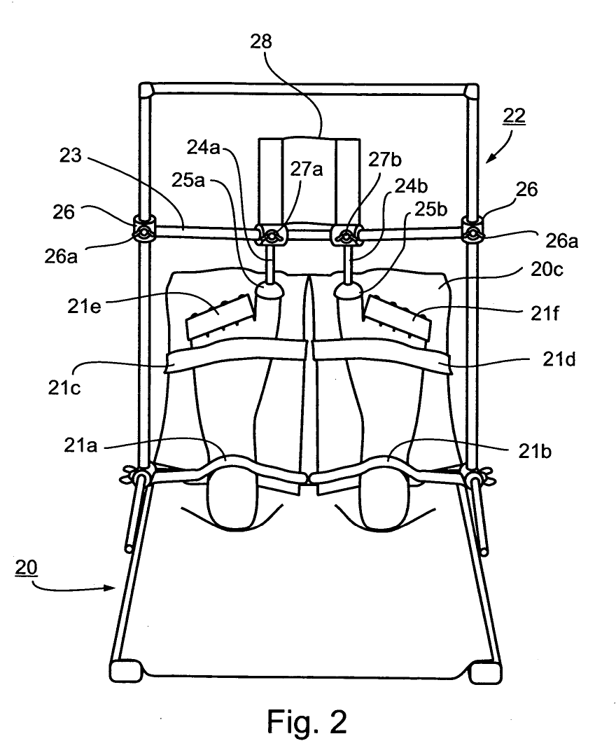

FIG. 2 illustrates an improved apparatus constructed in accordance with the present invention which overcomes the problems mentioned above encountered by the FIG. 1 prior art apparatus when used for evaluating nerve impairment by measuring EHL motor power of the patient.

The apparatus illustrated in FIG. 2 includes a frame, generally designated 20, configured for receiving and immobilizing both lower legs and feet of the patient but permitting movement of the patient's two big toes BT. Frame 20 further includes one or more flexible straps 21a, 21b for immobilizing each of two lower legs of the patient received within channel 20a, 20b, flexible straps 21c, 21d for immobilizing the foot of each leg, and flexible straps 21e, 21f for immobilizing the toes of each foot except for the big toe BT.

As shown particularly in FIG. 2, the apparatus therein illustrated further includes a rack 22 for mounting two force sensors engageable with the two big toes BT of the patients legs. Rack 22 thus includes a horizontal bar 23 to overlie the big toes BT, and an arm 24a, 24b, depending from horizontal bar 23 in alignment with each big toe. The end of each depending arm 24a, 24b carries a force sensor 25a, 25b in contact with the nail of the respective big toe BT.

Horizontal bar 23 is vertically adjustable by sleeves 26 and wing nut 26a, carried at its opposite ends, to enable vertical adjustment of the bar for the respective patient. In addition, each of the depending arms 24a, 24b, carrying the respective force sensor 25a, 25b, includes adjusting elements 27a, 27b, respectively, enabling horizontal adjustment of the force sensor along bar 23, as well as rotational adjustment to enable each force sensor to firmly engage the nail of the respective big toe of the patient.

In the apparatus of FIG. 2, the force sensors 25a, 25b are not of the spring type, as in the prior art apparatus of FIG. 1 which measures the sensed force by a displacement of a spring; rather, force sensors 25a, 25b in FIG. 2 are of a type which require very little displacement of the two big toes BT for the effective range of the measurement. Preferably, force sensors 25a, 25b are each displaceable for less than 1.0 mm for the effective range of the measurement.

For example, force sensors 25a, 25b may be resistor elements which change their electrical resistances in accordance with the force applied. An example of such a resistance element is that supplied under the trademark “FlexiForce” (Reg. T.M.) by Tekscan Inc. of South Boston, Mass. The A201 sensor is an ultra-thin flexible printed circuit of 0.008 inches, (i.e., about 0.2 mm) in thickness. Such sensors are constructed as a lamination including two layers of substrate (e.g., a polyester) with a conductive material (e.g., silver) and a pressure-sensitive ink inbetween. Since the overall thickness of the sensor is approximately 0.2 mm before a compressive force is applied, the displacement of the compressive force is necessarily less than 0.2 mm for the effective range of the measurement.

It will be appreciated that other types of force sensors may be used which require very little displacement, preferably less than 1.0 mm, for the effective range of the measurement.

In the apparatus illustrated in FIG. 2, the force sensors 25a, 25b are force transducers which output electrical signals corresponding to the forces applied by the respective big toes BT. The signals outputted by the force sensors are fed to a control unit 28 carried by rack 22, wherein they are amplified and converted to digital form before being inputted into a data processor for further processing.

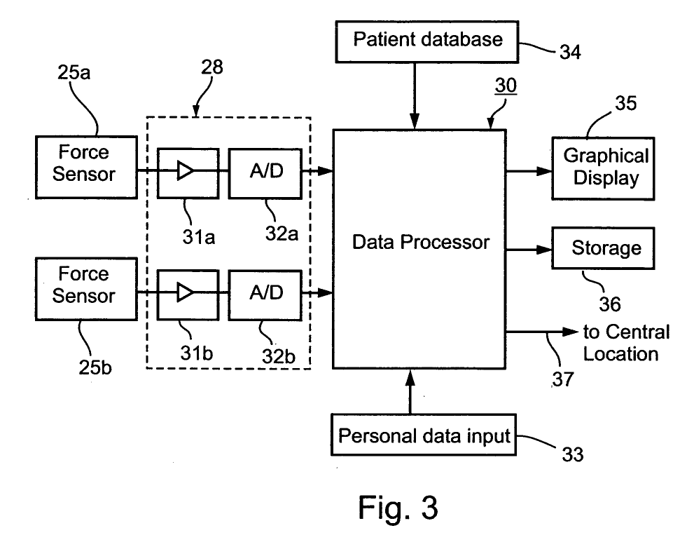

FIG. 3 is a block diagram of the overall electrical system including the data processor, therein generally designated 30. It will be seen from FIG. 3 that the outputs from force sensors 25a, 25b, after being amplified by amplifiers 31a, 31b and displayed by A/D converters 32a, 32b in control unit 28, are inputted into data processor 30. The data processor may also receive additional inputs, by a manual data input device 33, or by a patient datafile 34, personal to the respective patient. Dataprocessor 30 processes the inputted data and displays same in a graphical display 35.

Dataprocessor 30 further includes a storage device 36 for storing the data for future use, and an output device 37 for transmitting the data, e.g., to a central computer, for further processing, viewing, archiving, or other purpose.

The apparatus illustrated in FIGS. 2 and 3 may be used in the following manner to evaluate nerve impairment in a patient suffering from lumber discopathy:

While the patient is in a reclining position on a table, the two lower legs of the patient are placed within channel of the frame 20 with the soles of the two feet firmly contacting the end wall. The flexible belts 21a-21f are then applied to firmly immobilize both lower legs and feet of the patient while permitting movement of the patient's two big toes BT.

Horizontal bar 23 may then be vertically adjusted to the appropriate height for the respective patient, and the two depending arms 24a, 24b may be horizontally and rotatably adjusted so as to bring the force sensors 25a, 25b carried at their outer ends, into contact with the nails of the patient's two big toes BT. The data personal to the particular patient may then be inputted into the data processor 30 via input device 33 and/or database 34, if this has not been previously done.

After the apparatus has thus been set-up for the respective patient, the patient is requested to apply maximum force simultaneously by the two big toes in the extension direction, i.e., away from the sole of the respective foot, to the force sensors 25a, 25b. This maximum force capable of being applied by each of the two big toes is sensed by the force sensors 25a, 25b and converted to electrical signals which, after suitable amplification by amplifiers 31a, 31b and digitization by A/D converters 32a, 32b within the control unit 28, are introduced into the dataprocessor 30 for processing therein and for display in the graphical display 35. Such data may also be stored in the storage device 36 for use in the future tests. It may also be transmitted via the output port 37 to a central location for further processing, for viewing, for archiving, or for other purposes.

The apparatus is used as described above for measuring the maximum force capable of being applied by the patient's two big toes to the force sensors 25a, 25b in each of a plurality of separate actions over a predetermined time interval. The time interval is preferably at least one minute, and more preferably about two-three minutes. Such repeated testing provides an indication, not only of the maximum force capable of being applied by each big toe, but also of the extent of fatigue experienced by the two big toes when such actions are repeated over the predetermined time interval. Both the maximum initial force, and the extent of fatigue, are significant factors in evaluating nerve impairment in a patient suffering from lumber discopathy.

FIGS. 4-7 illustrate results of various tests performed on patients suffering from different types of lower back pain problems when the two legs are simultaneously tested by the apparatus illustrated in FIGS. 2 and 3 in the manner described above.

Thus, FIG. 4 illustrates the outputs of the force sensors 25a, 25b with respect to a patient having a healthy right leg and a slightly problematical left leg. This is indicated by the relatively equal amplitude of the output from the force sensor engaged by the big toe of the right leg (upper waveform), whereas the amplitude of the output from the force sensor engaged by the big toe of the left leg (lower waveform) became slightly reduced during the course of time.

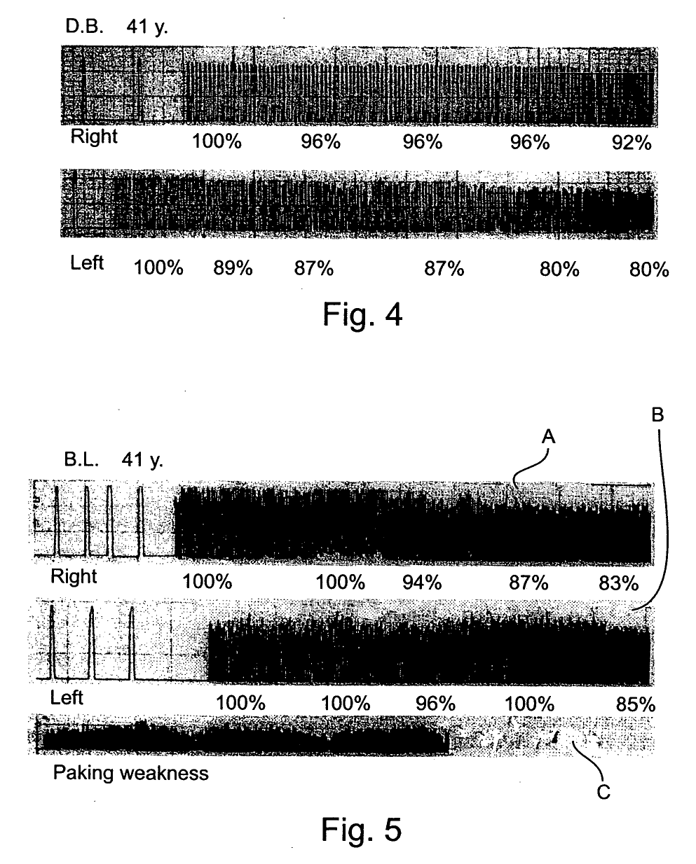

The waveforms illustrated in FIG. 5 demonstrate the advantages in measuring simultaneously the forces capable of being exerted by the big toes of both legs. Thus, the upper waveform A illustrates the output of the right leg sensor, and the middle waveform B illustrates the output of the left leg sensor when both legs are tested simultaneously by the apparatus illustrated in FIGS. 2 and 3 as described above. The lower waveform C on the other hand, illustrates the output of the left leg of the same patient when that leg is tested about ten minutes after the testing of the right leg. The substantial difference in the test results with respect to the left leg when in one case tested (waveform B) simultaneously with the right leg (waveforms A and B), and in the other case (waveform C) when tested ten minutes after testing of the right leg (waveforms A and C) is attributable to the different mental and physical conditions of the patient, at the two different times, in that the latter case introduces an additional variable to the examination, namely the actual or perceived fatigue of the patient at the time of the second examination.

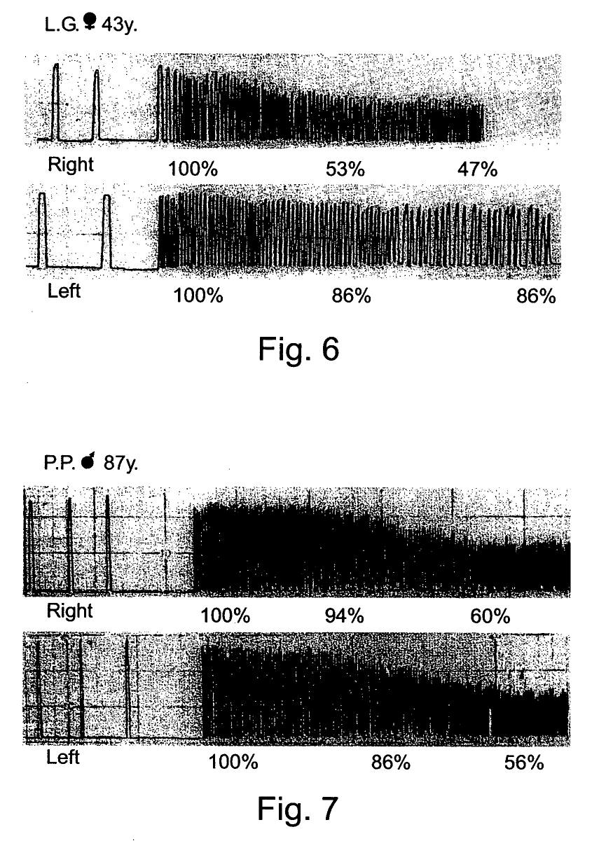

FIG. 6 illustrates the outputs of the force sensor with respect to a right leg which experiences a greater degree of fatigue in the course of the examination than the left leg, thereby indicating a problematical condition with respect to the right leg and a healthy condition with respect to the left leg.

FIG. 7 illustrates the outputs of the force sensors 25a, 25b, with respect to a patient exhibiting fatigue in both the right and left legs.

It will thus be seen that the use of the apparatus illustrated in FIGS. 2 and 3 in the manner described above provides a number of important advantages over the prior art apparatus of FIG. 1 for evaluating nerve impairment in a patient suffering from lumber discopathy. Thus, by measuring the forces capable of being exerted by the two big toes in extension simultaneously, not only was the examination time substantially decreased, but also more accurate and consistent results were obtained since it avoided the possibility of actual or perceived fatigue during a second examination. It also avoided introducing into the second examination the possibility of the patient's experience during the first examination influencing the results of the second examination. In addition, since the force sensors used do not require substantial movements of the big toes, more accurate results are obtainable particularly with respect to elderly patients with arthritic conditions which tend to produce pain and reduce cooperation when repeated motions are required. Further, since the data is outputted electronically, rather than mechanically, this data can be easily processed, displayed, and stored for future comparison purposes, for example.

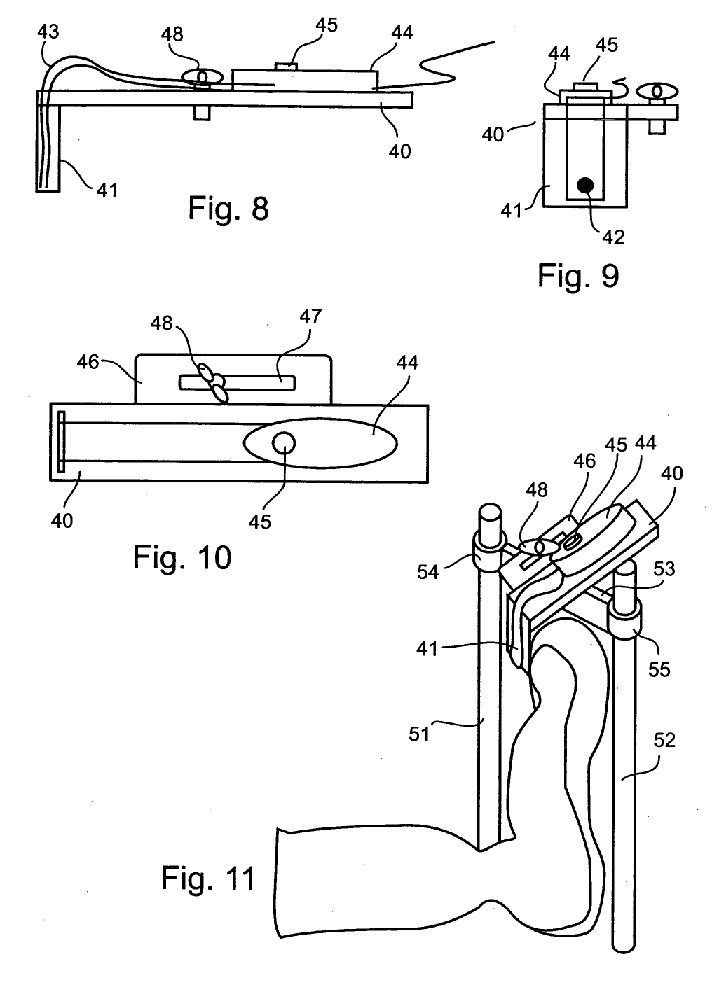

FIGS. 8-10 illustrate the construction of a force sensor unit which may be used with respect to each of the immobilized feet of the patient; and FIG. 11 illustrates the manner of adjustably mounting the force sensor unit with respect to the respective patient's foot.

Thus, as seen in FIGS. 8-10, each force sensor unit includes a base 40 having an end wall 41 rigidly secured to, and depending from, one end of the base. A sensor pad 42 is carried on the inner face of end wall 41. Sensor pad 42 is connected by electrical wires 43 to a housing 44 carried on the upper face of base 40 and housing the electrical circuit of the sensor, as described for example in the block diagram of FIG. 3. Housing 44 further includes one or more buttons or keys, as shown schematically at 45, for inputting data and/or for controlling the operation of the sensor.

Base 40 further includes a lateral extension 46 formed with a slot 47 for receiving an adjusting fastener 48, e.g., a butterfly screw, to enable adjustment of base 40, and particularly of sensor pad 42, relative to the respective big toe of the patient.

FIG. 11 illustrates the manner of mounting and adjusting the force sensor unit of FIGS. 8-10 with respect to the patient's big toe. Thus, as shown in FIG. 11, the apparatus frame includes a pair of uprights 51, 52, straddling the respective immobilized foot of the patient, and a crossbar 53 adjustably mounted with respect to uprights 51, 52 by slidable mounting elements 54, 55. Crossbar 53 carries a socket or bore (not shown) receiving threaded fastener 48, to enable adjusting of base 40, and thereby of sensor pad 42 carried by the base end wall 41, with respect to the patient's big toe to be examined. Thus, by loosening fastener 48, base 40, and thereby sensor pad 42 carried by end wall 41 of the base, may be adjusted towards and away from the patient's big toe while the patient is in a reclining position and while the patient's lower leg is immobilized, as described above with respect to FIG. 2.

Sensor pad 42 is also of the type requiring very little displacement for the effective operating range of the measurement. Preferably, each pressure pad 42 is of a compressible resistor element which changes its electrical resistance in accordance with the compressor force applied, and is displaceable for less than 1.0 mm for the effective operating range of the measurement.

In all other respects, the force sensor unit illustrated in FIGS. 8-11 may be used in the same manner as described above with respect to FIGS. 2 and 3.

While the invention has been described with respect to one preferred embodiment, it will be appreciated that this is set forth merely for purposes of example, and that many variations may be made. For example, other types of force sensors may be used which require a relatively small displacement for measuring force, e.g., strain-gauge type sensors. Many other variations, modifications and applications of the invention will be apparent.

What is claimed is:

1. A method of evaluating motor nerve impairment in a patient suffering from lower lumber discopathy, comprising:
immobilizing both lower legs and feet of the patient except for the patient's two big toes;
measuring the maximum forces capable of being applied simultaneously by the patient's two big toes in extension;
and utilizing said measurements for evaluating said motor nerve impairment.

2. The method according to claim 1, wherein said maximum forces are measured by force sensors which require very little displacement of the two big toes for the effective operating range of the measurement.

3. The method according to claim 2, wherein said force sensors are displaceable for less than 1.0 mm for the effective range of the measurement.

4. The method according to claim 2, wherein said force sensors are compressible resistor elements which change their electrical resistances in accordance with the compressive force applied.

5. The method according to claim 1, wherein said measuring operation measures the maximum forces capable of being applied simultaneously by the patient's two big toes only in extension.

6. The method according to claim 1, wherein all the toes of the patient are also immobilized except for the patient's big toes.

7. The method according to claim 6, wherein both lower legs, feet and all the toes of the patient except for the two big toes are immobilized by flexible straps.

8. The method according to claim 1, wherein said maximum forces capable of being applied by the patient's two big toes are measured in each of a plurality of separate actions over a predetermined time interval of at least one minute.

9. The method according to claim 1, wherein said force sensors are force transducers which output electrical signals corresponding to the forces applied by the respective toes.

10. The method according to claim 9, wherein said method also includes the additional operations of storing, processing and displaying the outputs of the force sensors.

11. Apparatus for evaluating motor nerve impairment in a patient suffering from lower lumber discopathy, comprising:
a frame configured for receiving and immobilizing both lower legs and feet of the patient except for the patient's two big toes;
and two force sensors carried by said frame at locations such that each force sensor is aligned with and engaged by one of the big toes of the patient, when the lower legs and feet of the patient are immobilized on said frame, for measuring the maximum forces capable of being simultaneously applied by the two big toes in extension.

12. The apparatus according to claim 11, wherein said force sensors are of a type requiring very little displacement for the effective operating range of the measurement.

13. The apparatus according to claim 12, wherein said force sensors are each displaceable for less than 1.0 mm for the effective operating range of the measurement.

14. The apparatus according to claim 12, wherein said force sensors are compressible resistor elements which change their electrical resistance in accordance with the compressive force applied.

15. The apparatus according to claim 11, wherein said frame includes:
two parallel spaced channels for receiving the two lower legs of the patient, and an end wall at one end of the two parallel spaced channels; said end wall extending substantially perpendicularly to said channels for contacting the soles of the two feet of the patient when the patient's lower legs are received within said channels;
said two force sensors being carried by said end wall so as to be in alignment with and engaged by the patient's two big toes when the patient's legs are received within said channels.

16. The apparatus according to claim 15, wherein said end wall is of a size to contact also the toes of the patient's two feet, and includes straps for immobilizing all the patient's toes except for the two big toes.

17. The apparatus according to claim 11, wherein said force sensors are force transducers which output electrical signals corresponding to the forces applied by the respective toes.

18. The apparatus according to claim 17, wherein said apparatus further comprises a data processor for processing the outputs of said two force transducers and for providing an indication of said nerve impairment.

19. The apparatus according to claim 11, wherein each of said force sensors includes:
a base adjustably mounted on said frame to overlie the respective foot of the patient when the patient is in a reclining position;
an end wall rigidly secured to one end of said base to depend therefrom and to be located just forwardly of the big toe of the patient when in the reclining position;
and a sensor pad carried on the inner face of said end wall in alignment with the big toe of the patient.

20. The apparatus according to claim 19, wherein said base is adjustably mounted to said frame by a lateral extension of said base formed with a slot receiving an adjusting screw, enabling adjustment of said sensor pad with respect to the patient's big toe.

21. Apparatus for evaluating motor nerve impairment in a patient suffering from lower lumber discopathy, comprising:
a frame configured for receiving and immobilizing at least one lower leg and foot of the patient except for the patient's big toe;
a force sensor carried by said frame in alignment with, and engaged by, the big toe of the immobilized leg and foot of the patient for measuring the maximum force capable of being applied by the big toe in extension;
said force sensor including a base adjustably mounted on said frame to overlie the immobilized foot of the patient when the patient is in a reclining position;
an end wall rigidly secured to one end of said base to depend therefrom and to be located just forwardly of the big toe of the patient's immobilized foot; and
a sensor pad carried on the inner face of said end wall in alignment with the big toe of the immobilized foot;
said base being adjustably mounted to said frame by a lateral extension of the base formed with a slot receiving an adjusting screw enabling adjustment of said sensor pad with respect to the big toe of the patient's immobilized foot.

22. The apparatus according to claim 21, wherein said force sensor is of a type requiring very little displacement for the effective operating range of the measurement.

23. The apparatus according to claim 22, wherein said force sensor is displaceable for less than 1.0 mm for the effective operating range of the measurement.

24. The apparatus according to claim 22, wherein said force sensor is a compressible resistor element which changes its electrical resistance in accordance with the compressive force applied.
